(19) United States
(12) Patent Application Publication
(10) Pub. No.: US 2019/0224177 A1
(43) Pub. Date: Jul. 25, 2019

(54) STABLE LIQUID COMPOSITIONS OF SODIUM PICOSULFATE

(71) Applicant: LUPIN INC., Somerset, NJ (US)

(72) Inventors: Bharat Kirthivasan, Somerset, NJ (US); Sabarinath Shankar, Somerset, NJ (US); James Garegnani, Somerset, NJ (US); Arjun Muthukkaruppan, Somerset, NJ (US)

(21) Appl. No.: 16/214,346

(22) Filed: Dec. 10, 2018

Related U.S. Application Data
(60) Provisional application No. 62/620,552, filed on Jan. 23, 2018.

(51) Int. Cl. A61K 31/4402; A61K 33/08; A61K 31/194; A61K 47/12; A61K 47/26; A61K 47/36; A61K 9/08

(52) U.S. Cl. CPC A61K 31/4402; A61K 33/08; A61K 31/194; A61K 9/08; A61K 47/26; A61K 47/36; A61K 47/12

(57) ABSTRACT

A stable pharmaceutical liquid composition of sodium picosulfate, magnesium oxide, citric acid, comprising one or more stabilizers selected from organic acid or its derivatives thereof selected from maleic acid, sodium gluconate, formic acid, sodium alginate, oxalic acid; ammonium chloride or a combination thereof is provided. The present invention also relates to process of making and using such a composition.

### FIELD OF INVENTION

The present invention provides a stable pharmaceutical liquid composition of sodium picosulfate, magnesium oxide, citric acid, comprising one or more stabilizers selected from organic acid or its derivatives thereof selected from maleic acid, sodium gluconate, formic acid, sodium alginate, oxalic acid; ammonium chloride or a combination thereof and process of making and using such a composition.

### BACKGROUND OF INVENTION

Pharmaceutical products containing picosulfate in the form of sodium picosulfate can be used to treat constipation or for the clearance of the bowel prior to X-ray examination, endoscopy or surgery. Particularly useful are products that contain sodium picosulfate in combination with magnesium oxide and anhydrous citric acid, which together in solution form magnesium citrate, an osmotic laxative with a powerful cathartic effect. Examples of such formulations are sold under the trade name PREPOPIK®. These products contain sodium picosulfate along with magnesium oxide and citric acid in the form of a solid that must be dissolved to be taken orally by the patient and provides a strong laxative that is easily palatable. The products are particularly effective to prepare patients for colonoscopy. In particular, the products include citric acid, magnesium oxide, and sodium picosulfate, as active ingredients, along with KHCO₃, sodium saccharin, and flavoring (e.g., orange flavor).

The existing products containing sodium picosulfate in combination with magnesium oxide and citric acid do, however, suffer from disadvantages. One is that the formulations do not dissolve immediately. For example, the patient instruction sheet for the PREPOPIK® product instructs the patient to pour the solid contents of a packet of the preparation in 5 fluid ounces (150 mL) of water in a cup, and to stir the resulting mixture for 2-3 min. before drinking the entire contents of the cup. If the patient fails to follow the procedure precisely, e.g., by failing to stir the contents for the full 2-3 min. before consumption, there is a risk that the product will not be fully dissolved and that the patient will receive less than a full dose of the product, and that the product will therefore not be as effective as intended.

To overcome the disadvantages described above, it would be desirable to be able to supply formulations containing picosulfate and magnesium citrate (MgO/citric acid) in liquid form, ready for consumption by the patient. However, dissolution of existing formulations containing sodium picosulfate and magnesium citrate leads to compositions that are unstable when stored or allowed to stand, with precipitates containing magnesium salts being formed from the solution.

PCT application no. WO2015/141897 discusses liquid pharmaceutical compositions containing sodium picosulfate, magnesium oxide, and citric acid. Precipitation was delayed at low pH (4.1), but decomposition occurred to form the known mono-sulfate hydrolysis product Sodium Picosulfate Related Compound ‘A’ ([(4-hydroxyphenyl)(pyridin-2-yl)methyl]phenyl sodium sulfate). Malic acid could be used to inhibit precipitation at pH 4.7-5.1, but other carboxylic acids like maleic acid were ineffective.

Liquid composition of sodium picosulfate, magnesium oxide and citric acid is recently approved as CLENPIQ® having malic acid as an agent providing stability wherein substance A related to sodium picosulfate (4-[(pyridine-2-yl)(4-hydroxyphenyl)methyl]phenyl sodium sulfate) may be generated at 2.0 wt. % or less.

U.S. application Ser. No. 15/238,408 discloses liquid composition of sodium picosulfate and magnesium citrate wherein composition is stabilized by precipitation inhibitor like carboxylic acid, ammonium salt or soluble anionic polymer.

U.S. application Ser. No. 15/643,727 discloses liquid composition of sodium picosulfate with antioxidant.

Although attempts were made in the past there is still need to develop physically and chemically stable composition of sodium picosulfate.

### SUMMARY OF THE INVENTION

The main aim of the invention is to provides a stable pharmaceutical liquid composition of sodium picosulfate, magnesium oxide, citric acid, comprising one or more stabilizers selected from organic acid or its derivatives thereof selected from maleic acid, sodium gluconate, formic acid, sodium alginate, oxalic acid; ammonium chloride or a combination thereof and process of making and using such a composition.

In one embodiment, a pharmaceutical liquid composition comprising sodium picosulfate, magnesium oxide, citric acid and one or more stabilizers selected from organic acid or its derivatives selected from maleic acid, sodium gluconate, formic acid, sodium alginate, oxalic acid; ammonium chloride or a combination thereof. In preferred embodiment the liquid compositions are stable over verity of storage conditions. In one embodiment, a stable pharmaceutical liquid composition comprising sodium picosulfate, magnesium oxide, citric acid and maleic acid.

In another embodiment, a stable pharmaceutical liquid composition comprising sodium picosulfate, magnesium oxide, citric acid and formic acid.

In another embodiment, a stable pharmaceutical liquid composition comprising sodium picosulfate, magnesium oxide, citric acid and oxalic acid.

In another embodiment, a stable pharmaceutical liquid composition comprising sodium picosulfate, magnesium oxide, citric acid, sodium gluconate, ammonium chloride and sodium alginate.

In another embodiment, a pharmaceutical liquid composition of sodium picosulfate, magnesium oxide, citric acid, comprising one or more organic acid or its derivatives thereof selected from maleic acid, sodium gluconate, formic acid, ammonium chloride, sodium alginate, oxalic acid or a combination thereof and process of making and using such a composition. In preferred embodiment, pharmaceutical compositions are stable.

In another embodiment, pharmaceutical liquid compositions are physically and chemically stable when stored under different storage conditions.

In another embodiment, a pharmaceutical liquid composition of sodium picosulfate, magnesium oxide, citric acid, comprising one or more organic acid or its derivatives thereof selected from maleic acid, sodium gluconate, formic acid, ammonium chloride, sodium alginate, oxalic acid or a combination thereof wherein, substance A related to sodium picosulfate (4-[(pyridine-2-yl)(4-hydroxyphenyl)methyl]phenyl sodium sulfate) may be generated at 2.0 wt. % or less.

In another embodiment, a pharmaceutical liquid composition comprising sodium picosulfate, magnesium oxide, citric acid, maleic acid wherein, substance A related to sodium picosulfate (4-[(pyridine-2-yl)(4-hydroxyphenyl)methyl]phenyl sodium sulfate) may be generated at 2.0 wt. % or less.

In another embodiment, a pharmaceutical liquid composition comprises maleic acid in the range from about 4.1 g to 12.2 g per 160 mL of liquid composition.

In another embodiment, a stable pharmaceutical liquid composition comprising sodium picosulfate, magnesium oxide, citric acid, maleic acid and sodium gluconate.

In another embodiment, a stable pharmaceutical liquid composition comprising sodium picosulfate, magnesium oxide, citric acid, sodium gluconate, ammonium chloride and sodium alginate.

In another embodiment, a pharmaceutical liquid composition comprising sodium picosulfate, magnesium oxide, citric acid, maleic acid and sodium gluconate wherein, substance A related to sodium picosulfate (4-[(pyridine-2-yl)(4-hydroxyphenyl)methyl]phenyl sodium sulfate) may be generated at 2.0 wt. % or less.

In another embodiment, a stable pharmaceutical liquid composition comprises sodium gluconate in the range from about 6.8 g to 20.6 g per 160 mL of liquid composition. In another embodiment, a stable pharmaceutical liquid composition of sodium picosulfate, magnesium oxide and citric acid comprising maleic acid in the range from about 4.1 g to 12.2 g per 160 mL of liquid composition and sodium gluconate in the range from about 6.8 g to 20.6 g per 160 mL of liquid composition wherein, substance A related to sodium picosulfate (4-[(pyridine-2-yl)(4-hydroxyphenyl)methyl]phenyl sodium sulfate) may be generated at 2.0 wt. % or less.

In another embodiment, a pharmaceutical liquid compositions of inventions can be used for colon cleaning as a purgative for pretreatment at the time of surgery, colonoscopy or colon X-ray inspection.

A one-time dose of the pharmaceutical liquid composition may be different according to the content of an effective ingredient, but may range from 50 mL to 500 mL as a non-limiting example. In an exemplary embodiment, it may range from 100 mL to 300 mL or may range from 150 mL to 200 mL, but it is not limited thereto.

In another embodiment, a pharmaceutical liquid composition which is physically and chemically stable when stored.

In another embodiment, pharmaceutical liquid compositions when stored for 24 months, a content change of each ingredient may be within ±5.0 wt. % (weight percent) with respect to the weight of each ingredient, and impurities. Substance A related to sodium picosulfate (4-[(pyridine-2-yl)(4-hydroxyphenyl)methyl]phenyl sodium sulfate) may be generated at 2.0 wt. % or less. Further, the precipitate may not occur, or may occur at 5.0 vol. % (volume percent) or less.

In another embodiment, the pH of the stable pharmaceutical liquid compositions may be in the range from 4.7 to 5.7.

In another embodiment, a stable pharmaceutical liquid composition comprising sodium picosulfate, magnesium oxide, citric acid, sodium alginate, sodium gluconate and ammonium chloride; wherein the composition comprises sodium gluconate in the range from about 6.8 g to 20.6 g; sodium alginate in an amount of about 1.66 g and ammonium chloride in an amount of about 2.5 g per 160 ml of liquid composition; wherein the substance A related to sodium picosulfate (4-[(pyridine-2-yl)(4-hydroxyphenyl)methyl]phenyl sodium sulfate) may be generated at 2.0 wt. % or less.

The pharmaceutical liquid composition of the present invention may include a variety of excipients and purified water. The purified water is used in order to prepare the medicine in the form of liquid, and the excipients may be used for the excellent taste to increase the medication compliance and the stability of the pharmaceutical liquid composition.

For example, the excipients may include, but not limited to, a pH adjuster, a buffer, a stabilizer, a preservative, a chelating agent, an antioxidant, an antimicrobial, an air displacement agent, a sweetener and a fragrance ingredient.

The pH adjuster may be an alkalizing or an acidifying agent. In the case of the alkalizing agent, it is possible to adjust a reduction in pH due to the maleic acid and sodium gluconate.

As the alkalizing agent, for example, sodium hydroxide, potassium hydroxide, sodium bicarbonate, ammonia solution, potassium citrate, triethanolamine and sodium citrate and the like may be used, but it is not limited thereto. In an exemplary embodiment, sodium hydroxide, potassium hydroxide, sodium citrate, and the like may be used, but it is not limited thereto. As the acidifying agent, for example hydrochloric acid, sulfuric acid and the like may be used, but it is not limited thereto. Nitrogen or Carbon dioxide may be utilized as the air displacement agents but it is not limited thereto.

Many other variations of the present invention will be apparent to those skilled in the art and are meant to be within the scope of the claims appended hereto. The foregoing specification alludes to beliefs, hypothesis and conclusions of the inventors based on his experience in the field, the reports of others and experiments conducted and reported herein, and are provided for purposes of (possible) explanation only and are not meant to limit the invention in any manner whatsoever.

The following examples illustrate various aspects of the present invention, and are set forth to assist in understanding the invention. These examples should not be construed as specifically limiting the invention described and claimed herein. Variations of the invention, including the substitution of all equivalents now known or later developed, which would be within the purview of those skilled in the art, and changes in formulation or minor changes in experimental design, are considered to fall within the scope of the invention and appended claims.

### EXAMPLE 1

| | Amount/Unit | Amount (% |
|---|---|---|
| Ingredients | (160 mL) | Wt./mL) |
| Sodium Picosulfate | 0.010 | 0.00625 |
| Magnesium Oxide | 3.50 | 2.19 |
| Citric acid anhydrous | 12.00 | 7.50 |
| Maleic acid | 8.4 | 5.25 |
| Sodium gluconate | 12.3 | 7.69 |
| Acesulfame Potassium | 0.240 | 0.15 |
| Orange Flavor | 0.160 | 0.10 |
| Disodium Edetate | 0.080 | 0.05 |
| Sodium Benzoate | 0.100 | 0.0625 |
| Sodium Metabisulfite | 0.160 | 0.10 |
| Sucralose | 0.240 | 0.15 |
| Sodium Hydroxide | Q.S | Q.S to adjust |
| | | pH |
| Hydrochloric acid | Q.S | Q.S to adjust |
| | | pH |
| Purified Water | Q.S | 75.0 |
| Purified Water | Q.S | Q.S to 100% |
| Nitrogen | Trace | Trace |
| Total | 172 g/160 mL | 100% |

### Manufacturing Procedure:

1. Weigh Purified water in a container.
2. Add Acesulfame Potassium, Orange Flavor, Disodium Edetate, Sodium Benzoate, Sodium
Metabisulfite and Sucralose.
3. Add Magnesium Oxide, mix until complete dissolution.
4. Add Citric acid anhydrous, mix until complete dissolution.
5. Add Maleic acid and Sodium gluconate, into the solution. Mix until dissolved.
6. Adjust the pH using Sodium Hydroxide and Hydrochloric acid to be between pH 4.7 to 5.7.
7. Add Sodium Picosulfate. Mix until dissolved.
8. Filter the above solution through 100 mesh screen.
9. Make up the volume of each container to 160 mL, with purified water.
10. Nitrogen sparge the bulk solution.

### EXAMPLE 2

| | Amount/ | |
|---|---|---|
| Ingredients | Unit (160 mL) | Amount (%) |
| Sodium Picosulfate | 0.010 | 0.00625 |
| Magnesium Oxide | 3.50 | 2.19 |
| Citric acid anhydrous | 12.00 | 7.50 |
| Maleic acid | 8.4 | 5.25 |
| Acesulfame Potassium | 0.240 | 0.15 |
| Orange Flavor | 0.160 | 0.1 |
| Disodium Edetate | 0.080 | 0.05 |
| Sodium Benzoate | 0.100 | 0.0625 |
| Sodium Metabisulfite | 0.160 | 0.1 |
| Sucralose | 0.240 | 0.15 |
| Sodium Hydroxide | Q.S | Q.S to adjust |
| | | pH |
| Hydrochloric acid | Q.S | Q.S to adjust |
| | | pH |
| Purified Water | Q.S | 75.0 |
| Purified Water | Q.S | Q.S to 100% |
| Nitrogen | Trace | Trace |
| Total | 172 g/160 mL | 100% |

### Procedure:

1. Weigh Purified water in a container.
2. Add Acesulfame Potassium, Orange Flavor, Disodium Edetate, Sodium Benzoate,
Sodium Metabisulfite and Sucralose.
3. Add Magnesium Oxide, mix until complete dissolution.
4. Add Citric acid anhydrous, mix until complete dissolution.
5. Add Maleic acid into the solution. Mix until dissolved.
6. Adjust the pH using Sodium Hydroxide and Hydrochloric acid to be between pH 4.7 to 5.7.
7. Add Sodium Picosulfate. Mix until dissolved.
8. Filter the above solution through 100 mesh screen.
9. Make up the volume of each container to 160 mL, with purified water.
10. Nitrogen sparge the bulk solution.

### EXAMPLE 3

| | Amount/Unit | |
|---|---|---|
| Ingredients | (160 mL) | Amount (%) |
| Sodium Picosulfate | 0.010 | 0.00625 |
| Magnesium Oxide | 3.50 | 2.19 |
| Citric acid anhydrous | 12.00 | 7.50 |
| Sodium Gluconate | 12.3 | 7.69 |
| Acesulfame Potassium | 0.240 | 0.15 |
| Orange Flavor | 0.160 | 0.1 |
| Disodium Edetate | 0.080 | 0.05 |
| Sodium Benzoate | 0.100 | 0.0625 |
| Sodium Metabisulfite | 0.160 | 0.1 |
| Sucralose | 0.240 | 0.15 |
| Sodium Hydroxide | Q.S | Q.S to adjust |
| | | pH |
| Hydrochloric acid | Q.S | Q.S to adjust |
| | | pH |
| Purified Water | Q.S | 75.0 |
| Purified Water | Q.S | Q.S to 100% |
| Nitrogen | Trace | Trace |
| Total | 172 g/160 mL | 100% |

### Procedure:

1. Weigh Purified water in a container.
2. Add Acesulfame Potassium, Orange Flavor, Disodium Edetate, Sodium Benzoate,
Sodium Metabisulfite and Sucralose.
3. Add Magnesium Oxide, mix until complete dissolution.
4. Add Citric acid anhydrous, mix until complete dissolution.
5. Add Maleic acid into the solution. Mix until dissolved.
6. Adjust the pH using Sodium Hydroxide and Hydrochloric acid to be between pH 4.7 to 5.7.
7. Add Sodium Picosulfate. Mix until dissolved.
8. Filter the above solution through 100 mesh screen.
9. Make up the volume of each container to 160 mL, with purified water.
10. Nitrogen sparge the bulk solution.

### EXAMPLE 4

| | Amount/Unit | |
|---|---|---|
| Ingredients | (160 mL) | Amount (%) |
| Sodium Picosulfate | 0.010 | 0.00625 |
| Magnesium Oxide | 3.50 | 2.19 |
| Citric acid anhydrous | 12.00 | 7.50 |
| Sodium Gluconate | 12.0 | 7.50 |
| Ammonium Chloride | 2.5 | 1.56 |
| Sodium Alginate | 1.66 | 1.04 |
| Acesulfame Potassium | 0.200 | 0.15 |
| Orange Flavor | 0.080 | 0.10 |
| Disodium Edetate | 0.210 | 0.05 |
| Sodium Benzoate | 0.090 | 0.0625 |
| Sodium Metabisulfite | 0.320 | 0.10 |
| Sucralose | 0.200 | 0.15 |
| Sodium Hydroxide | Q.S | Q.S to adjust |
| | | pH |
| Hydrochloric acid | Q.S | Q.S to adjust |
| | | pH |
| Purified Water | Q.S | 75.0 |
| Purified Water | Q.S | Q.S to 100% |
| Nitrogen | Trace | Trace |
| Total | 172 g/160 mL | 100% |

### Procedure:

1. Weigh Purified water in a container.
2. Add Acesulfame Potassium, Orange Flavor, Disodium Edetate, Sodium Benzoate,
Sodium Metabisulfite and Sucralose.
3. Add Magnesium Oxide, mix until complete dissolution.
4. Add Citric acid anhydrous, mix until complete dissolution.
5. Add Sodium gluconate, Ammonium chloride and Sodium alginate into the solution. Mix until dissolved.
6. Adjust the pH using Sodium Hydroxide and Hydrochloric acid to be between pH 4.7 to 5.7.
7. Add Sodium Picosulfate. Mix until dissolved.
8. Filter the above solution through 100 mesh screen.
9. Make up the volume of each container to 160 mL, with purified water.
10. Nitrogen sparge the bulk solution.

### Stability Studies:

Pharmaceutical liquid compositions of invention Example 1-4 were tested under variety of conditions with or without nitrogen sparging. The Reference example 1 was prepared by similar process and components as described in Example 1 except addition of malic acid in place of maleic acid. The Reference example 2 was prepared by similar process and components as described in Example 4 except addition of malic acid in place of sodium gluconate, sodium alginate and ammonium chloride. The results obtained are disclosed in Table 1. It was observed that Example 1-4 of the invention are stable under variety of conditions compared to Reference Examples 1 and 2.

TABLE 1
Stability Data

| | | | | | Single | |
|---|---|---|---|---|---|---|
| | | | Imp | Imp | largest | |
| Example | pH | Condition | A | B | Unknown | Total |
| Example 2 | 4.2 | Initial | 0.374 | ND | 0.362 | 0.736 |
| (without Nitrogen | 4.7 | Initial | 0.227 | ND | 0.315 | 0.542 |
| sparging) | 5.2 | Initial | 0.237 | ND | 0.400 | 0.637 |
| Example 3 | 4.2 | Initial | 0.227 | ND | 0.263 | 0.490 |
| (without Nitrogen | 4.7 | | 0.290 | ND | 0.439 | 0.729 |
| sparging) | 5.2 | | 0.132 | ND | 0.317 | 0.449 |
| Example 1 | 4.2 | Initial | 0.336 | ND | 0.254 | 0.590 |
| (without Nitrogen | | Freeze thaw | 0.315 | ND | 0.209 | 0.594 |
| sparging) | 4.7 | Initial | 0.167 | ND | 0.217 | 0.384 |
| | | Freeze thaw | 0.162 | ND | 0.232 | 0.394 |
| | 5.2 | Initial | 0.145 | ND | 0.286 | 0.431 |
| | | Freeze thaw | 0.131 | ND | 0.297 | 0.428 |
| Reference | 4.2 | Freeze thaw | 1.060 | ND | 1.552 | 2.612 |
| Example 1 | | 2M ACC | 4.139 | 0.040 | 1.998 | 6.275 |
| Malic acid (without | | 3M ACC | 5.669 | 0.078 | 2.478 | 8.550 |
| Nitrogen sparging) | 4.9 | 2M ACC | 1.248 | ND | 2.678 | 4.146 |
| | | 3M ACC | 1.518 | ND | 3.771 | 5.562 |
| | 5.3 | 2M ACC | 0.403 | ND | 1.292 | 1.816 |
| | | 3M ACC | 0.455 | ND | 1.798 | 2.382 |
| Reference | 5.0 | Initial | 0.062 | ND | 0.212 | 0.355 |
| Example 2 | | 2W 55C | 0.642 | ND | 1.036 | 1.755 |
| Malic acid (with | | 1M ACC | 0.290 | ND | 0.624 | 1.009 |
| Nitrogen | | 2M ACC | 0.451 | ND | 0.875 | 1.326 |
| sparging) - | | 3M ACC | 0.598 | ND | 1.145 | 1.742 |
| Similar as | | 1M CRT | 0.134 | ND | 0.369 | 0.608 |
| Example 4 | | 6W RT | 0.123 | ND | 0.309 | 0.564 |
| | | 3M CRT | 0.174 | ND | 0.439 | 0.613 |
| Example 4 | 5.3 | Initial | ND | ND | 0.184 | 0.294 |
| | | Without | ND | ND | 0.195 | 0.263 |
| | | Nitrogen | | | | |
| | | sparging | | | | |
| | | 2W 55C | 0.179 | ND | 0.263 | 0.737 |
| | | Freeze-thaw | ND | ND | 0.194 | 0.437 |
| | | 3M CRT | 0.045 | ND | 0.301 | 0.368 |
| | | 3M ACC | 0.186 | ND | 0.472 | 0.671 |

1. A stable pharmaceutical liquid composition comprising sodium picosulfate, magnesium oxide, citric acid and one or more stabilizers selected from organic acid or its derivatives selected from maleic acid, sodium gluconate, formic acid, sodium alginate, oxalic acid; ammonium chloride or a combination thereof.

2. The stable pharmaceutical liquid composition, according to claim 1, wherein the stabilizing agent is organic acid or its derivatives.

3. The stable pharmaceutical liquid composition, according to claim 2, wherein the organic acid or its derivative is maleic acid.

4. The stable pharmaceutical liquid composition according to claim 2, wherein the organic acid or its derivative comprises sodium gluconate and maleic acid.

5. The stable pharmaceutical liquid composition according to claim 2, wherein the organic acid or its derivative comprises sodium gluconate and sodium alginate.

6. The stable pharmaceutical liquid compositions according to claim 5, further comprises ammonium chloride as stabilizing agent.

7. The stable pharmaceutical liquid composition according to claim 6, comprises sodium gluconate in the range from about 6.8 g to 20.6 g per 160 ml of liquid composition.

8. The stable pharmaceutical liquid composition according to claim 7, comprises sodium alginate in an amount of about 1.66 g per 160 ml of liquid composition.

9. The stable pharmaceutical liquid composition according to claim 8, comprises ammonium chloride in an amount of about 2.5 g per 160 ml of liquid composition.

10. The stable pharmaceutical liquid composition according to claim 9, The stable pharmaceutical liquid composition according to claim 10, further comprises one or more excipients selected from pH adjuster, a buffer, a stabilizer, a preservative, a chelating agent, an antioxidant, an antimicrobial, an air displacement agent, a sweetener and a fragrance ingredient.

11. The stable pharmaceutical liquid composition according to claim 11, used for colon cleansing as a purgative for pretreatment at the time of surgery, colonoscopy or colon X-ray inspection.
